(19) United States
(12) Patent Application Publication
(10) Pub. No.: US 2013/0211467 A1
(43) Pub. Date: Aug. 15, 2013

(54) CONNECTOR AND FASTENER SYSTEM

(71) Applicant: Charles Anthony Dickinson, Bartlett, TN (US)

(72) Inventor: Charles Anthony Dickinson, Bartlett, TN (US)

(73) Assignee: WARSAW ORTHOPEDIC, INC., Warsaw, IN (US)

(21) Appl. No.: 13/371,145

(22) Filed: Feb. 10, 2012

(51) Int. Cl. A61B 17/84

(52) U.S. Cl. 606/328

(57) ABSTRACT

A spinal construct is provided which includes a bone penetrating member, a receiver and a spherical connector. The bone penetrating member has a head and a tip. The receiver extends along a longitudinal axis between a proximal end portion and a distal end portion. The proximal end portion is configured to receive an elongated member, such as a vertebral rod. The distal end portion of the receiver includes an opening configured for receiving a spherical connector. The spherical connector is configured to receive the head of the bone penetrating member. The spherical connector can have a truncated spherical shape and contains an upper portion, a middle portion and a lower portion with an axial bore through it.

### TECHNICAL FIELD

The present disclosure generally relates to medical devices, systems and methods for the treatment of musculoskeletal disorders, and more particularly to a spinal construct that employs a spherical connector for a universal attachment system to provide stabilization of vertebrae.

### BACKGROUND

Spinal disorders such as degenerative disc disease, disc herniation, osteoporosis, spondylolisthesis, stenosis, scoliosis, kyphosis, and other curvature abnormalities, tumor, and fracture may result from factors including trauma, disease and degenerative conditions caused by injury and aging. Spinal disorders typically result in symptoms including pain, nerve damage, and partial or complete loss of mobility. For example, after a disc collapse, severe pain and discomfort can occur due to the pressure exerted on nerves and the spinal column.

Non-surgical treatments, such as medication, rehabilitation and exercise can be effective. However, these treatments may fail to relieve the symptoms associated with these disorders. Surgical treatment of these spinal disorders includes discectomy, laminectomy, fusion and implantable prosthetics. As part of these surgical treatments, spinal constructs affixed to vertebral rods are often used to provide stability to a treated region. During surgical treatment, one or more rods may be attached via spinal constructs to the exterior of two or more vertebral members.

Any surgical operation, by nature, is a delicate procedure; operations proximate to the spinal column are even more delicate and demanding. In addition to the expected surgical procedures, the surgeon must force the spinal column and individual vertebra into alignment. During this procedure or immediately thereafter, the surgeon must position the fixation elements, assemble the spinal rod system, secure the spinal rod system to the vertebrae, and then tighten the connections in the entire system so no further movement occurs. Assembly of the spinal rod system can be very difficult, especially when the components are coated with body fluids. A more “user friendly” spinal fixation system that could be assembled quickly and reliably in the operating room would be a great benefit to both surgeons and their patients.

Typical spinal implant or bone stabilization systems utilize a rod as the support and stabilizing element. In such a system, a series of two or more spinal constructs, for example, bone fasteners, are inserted into two or more vertebrae to be instrumented. A rod or other stabilizing device is then placed within or attached to the head(s) of the bone fastener(s), or is placed within a coupling device that links the rod and the head(s) of the bone fastener(s). The connections between these multiple components are then secured, thereby fixing the supporting construct to multiple levels in the spinal column.

To advance the state of orthopedic implants, enhancement to such bone stabilization systems are believed desirable, and are addressed herein. This disclosure describes an improvement over these prior art technologies.

### SUMMARY

Accordingly, a spinal construct is provided which includes a bone penetrating member having a head and a tip opposite the head, a receiver extending along a longitudinal axis between a proximal end portion and a distal end portion of the receiver. The proximal end portion of the receiver is configured to receive an elongated member and the distal end portion comprising an opening for receiving a spherical connector, which is configured to receive the head of the bone penetrating member.

In an embodiment, the spherical connector comprises a truncated spherical shape. The spherical connector includes an upper portion, a middle portion, a lower portion and an axial bore extending through it. The upper portion of the spherical connector also contains external ridges configured to fit into the distal end portion of the receiver. In various embodiments, the upper portion and the lower portion of the spherical connector comprise a shape selected from a hexagon, a circle, an ellipse, a rectangle, a square or any other shape enabling the spherical connector to lock onto the head of a bone penetrating member. In other aspects, the middle portion of the spherical connector includes notches configured for receiving an inner locking ring.

In other embodiments, the receiver also contains an interface connector and a retaining member, the interface connector provided for retaining the upper portion of the spherical connector, and the retaining member for retaining the lower portion of the spherical connector.

In other embodiments, the head of the bone penetrating member is multi-axial which means that the receiver is configured to rotate freely about the head of the bone penetrating member prior to affixing an elongated member in the spinal construct, such as for example, a vertebral rod. In various embodiments the spinal construct contains a marker for indicating the position of the construct.

According to another aspect, the spinal construct comprises a bone penetrating member having a head and a tip opposite the head, a receiver extending along a longitudinal axis between a proximal end portion and a distal end portion of the receiver and a base having an upper end and a lower end. The base can be rotatably mounted to the distal end portion of the receiver, wherein the proximal end portion of the receiver is configured to receive an elongated member and (i) the base is configured to receive a spherical connector or (ii) the distal portion the receiver is configured to receive a spherical connector or (iii) the distal portion of the receiver and the base are configured to receive a spherical connector, the spherical connector configured to receive the head of the bone penetrating member.

In yet another aspect, a spinal construct is provided including a bone penetrating member having a head and a tip opposite the head, a receiver extending along a longitudinal axis between a proximal end portion and a distal end portion of the receiver. In this aspect the receiver includes an upper leg and a lower leg opposite the upper leg, the lower leg including a foot portion extending from one end thereof, the upper leg extending transversely to the longitudinal axis; an intermediate portion opposite the foot portion, the intermediate portion extending between the upper leg and the lower leg, the lower leg and the intermediate portion defining a cavity configured to receive a spherical connector, the spherical connector configured to receive the head of the bone penetrating member. The receiver also includes an upper passage portion extending through the receiver substantially perpendicular to the longitudinal axis into which a spinal rod can be placed.

### BRIEF DESCRIPTION OF THE DRAWINGS

The present disclosure will become more readily apparent from the specific description accompanied by the following drawings, in which:

FIG. 1 illustrates an enlarged perspective view of one particular embodiment of the spinal construct in accordance with the principles of the present disclosure;

FIG. 2 illustrates an enlarged perspective view the embodiment of the spinal construct illustrated in FIG. 1;

FIG. 3 illustrates an enlarged cross-sectional view along axis L₁ of the spinal construct shown in FIG. 1.

FIG. 4 illustrates an enlarged frontal cross-sectional view of the spinal construct illustrated in FIG. 1;

FIG. 5 illustrates an enlarged perspective view of another particular embodiment of the spinal construct in accordance with the principles of the present disclosure; and

FIG. 6 illustrates an enlarged perspective view of yet another particular embodiment of the spinal construct in accordance with the principles of the present disclosure.

Like reference numerals indicate similar parts throughout the figures. It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

The exemplary embodiments of spinal construct and related methods of use disclosed are discussed in terms of medical devices for the treatment of musculoskeletal disorders and more particularly, in terms of a spherical connector that provides a universal connection system to spine surgeons. The spinal construct described herein below allows the use of a singular bone screw component with multiple types of rod receivers thereby minimizing inventory while creating assemblies customized for a specific patient.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the present disclosure may be employed with other osteal and bone related applications, including those associated with diagnostics and therapeutics. It is contemplated that the disclosed bone fastener and system may be alternatively employed in a surgical treatment with a patient in a prone or supine position, and/or employs various surgical approaches to the spine, including anterior, posterior, posterior mid-line, medial, lateral, postero-lateral, and/or antero-lateral approaches, and in other body regions. The present disclosure may also be alternatively employed with procedures for treating the lumbar, cervical, thoracic and pelvic regions of a spinal column. The spinal construct and system and methods of the present disclosure may also be used on animals, bone models and other non-living substrates, such as, for example, in training, testing and demonstration.

The present disclosure may be understood more readily by reference to the following detailed description of the disclosure presented in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “superior” and “inferior” are relative and used only in the context to the other, and are not necessarily “upper” and “lower”.

Further, as used in the specification and including the appended claims, “treating” or “treatment” of a disease or condition refers to performing a procedure that may include administering one or more drugs to a patient in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

The components of the spinal construct can be fabricated from biologically acceptable materials suitable for medical applications, including metals, synthetic polymers, ceramics and bone material and/or their composites, depending on the particular application and/or preference of a medical practitioner. For example, the components of the spinal construct and universal attachment system, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of the spinal construct and universal attachment system may have material composites, including the above materials, to achieve various desired characteristics such as strength, rigidity, elasticity, compliance, biomechanical performance, durability and radiolucency or imaging preference. The components of the bone fastener system, individually or collectively, may also be fabricated from a heterogeneous material such as a combination of two or more of the above-described materials.

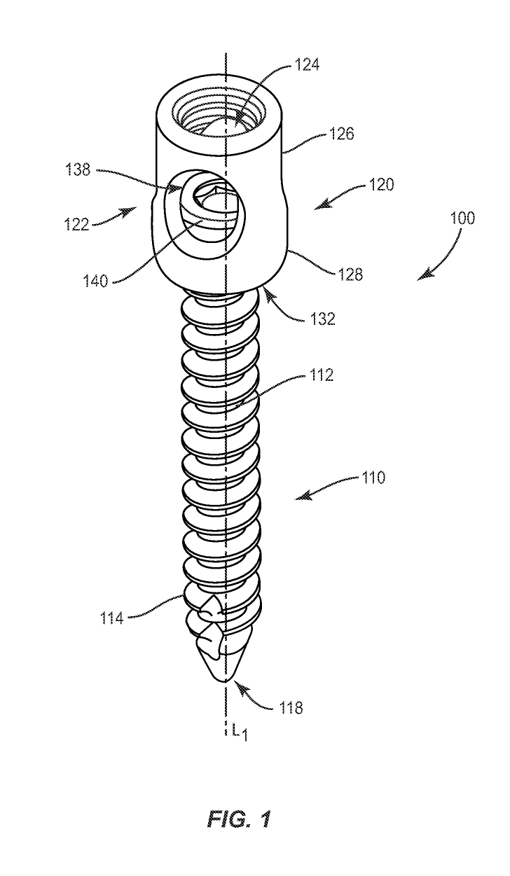

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications in the described devices, instruments, methods, and any further application of the principles of the disclosure as described herein are contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. The following discussion includes a description of a spinal construct and related methods of employing the bone fastener and system in accordance with the principles of the present disclosure. Alternate embodiments are also disclosed. Reference will now be made in detail to the exemplary embodiments of the present disclosure, which are illustrated in the accompanying figures. Turning now to FIGS. 1-6, there are illustrated components of a spinal construct and a universal bone attachment system in accordance with the principles of the present disclosure.

With reference to FIGS. 1-3, in an embodiment, there is provided a spinal construct 100 including a bone penetrating member 110 and a receiver 120. The bone penetrating member 110 includes a shank 112 having an outer surface 114, which in some embodiments is threaded to allow the bone penetrating member 110 to function as a bone screw, for example, a pedicle screw Shank 112 has a cylindrical shaft configuration, a head 116 and a tip 118. In some embodiments head 116 can be rounded and configured to fit within an interior cavity of distal end 128 of receiver 120 and contact interface connector 140 while allowing rotation and articulation of bone penetrating member 110. A set screw (not shown) secures a vertebral rod (not shown) to spinal construct 100. The spinal construct 100 may be part of a larger orthopedic system comprising a plurality of longitudinal members (e.g., rods, plates, etc.), a plurality of bone fasteners, and/or a plurality of connectors. In some embodiments, the spinal construct 100 is particularly suited for use in the spinal column. It will be understood that various types of fasteners or connectors (e.g., clamps) can be used in combination with the spinal construct 100 and vertebral rod.

Shank 112 defines a longitudinal axis L₁ and is configured for fixation with spinal vertebrae. It is contemplated that bone penetrating member 110 may include alternate bone fixation elements, such as, for example, a nail configuration, barbs, and/or expanding elements.

It is contemplated that bone penetrating member 110 can be variously dimensioned, for example, with regard to length, width, diameter and thickness. It is further contemplated that the respective cross-sectional geometry of member 110 may have various configurations, for example, round, oval, rectangular, irregular, consistent, variable, uniform and non-uniform. Bone penetrating member 110 may have a different cross-sectional area, geometry, material or material property such as strength, modulus or flexibility relative to shank 112.

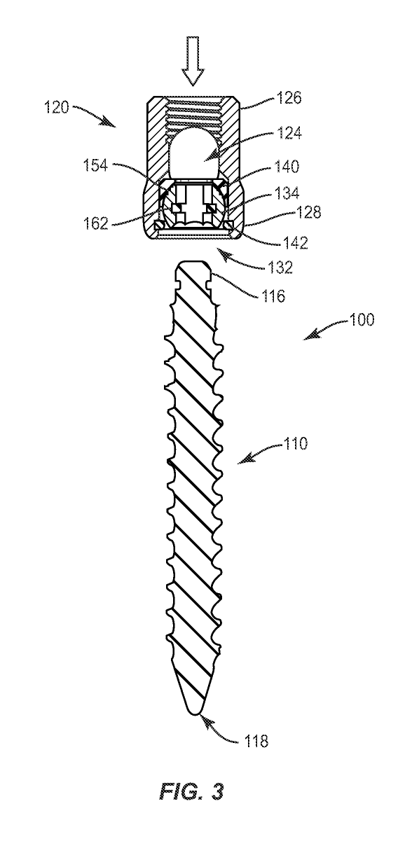

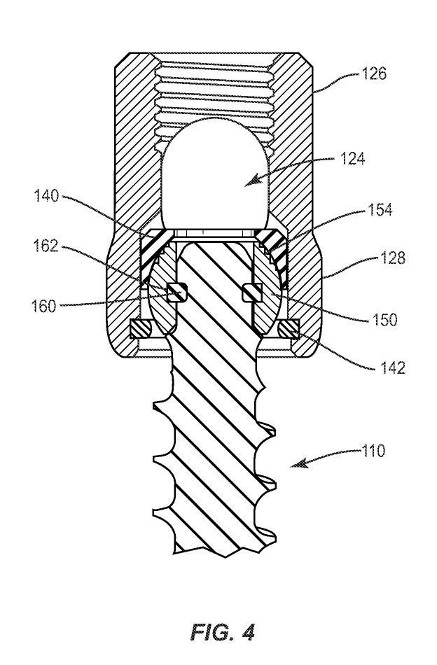

Spinal construct 100 further includes a receiver 120 for a vertebral rod. More particularly, receiver 120 includes a proximal end portion 126, a body 122, a lower passage portion 132 configured to receive the head of the bone fastener, and a distal end portion 128 centered about longitudinal axis L₁. Proximal end portion 126 includes a reduced size relative to distal end portion 128. As shown in FIG. 3, receiver 120 includes an upper passage portion 124 extending through proximal end portion 126 in communication with a receptacle 134 defined in distal end portion 128. At its upper end, distal end portion 128 includes an interface connector 140 configured for receiving and retaining the upper portion of spherical connector 150 (shown in FIG. 4) therein. In various embodiments, interface connector 140 is shaped as an annular jacket in contact with and adjacent spherical connector 150 (shown in FIG. 4). In some embodiments it is contemplated that interface connector 140 minimizes potential friction between the vertebral rod and the spherical connector 150 (shown in FIG. 4). At its lower end, distal end portion 128 includes a retaining member(s) 142. In some embodiments retaining member(s) 142 can be a washer or a ring. Receiver 120 also defines windows 138 in opposite sides thereof in communication with receptacle 134.

Proximal end portion 126 of receiver 120 defines upper passage portion 124 (also shown in FIG. 4) which is threaded and configured to receive and support vertebral rod, and a set screw, which can be rotated into receiver 120 to attach vertebral rod in place with spinal vertebrae. Distal end 128 portion defines receptacle 134, which is configured to receive spherical connector 150 (shown in FIG. 4).

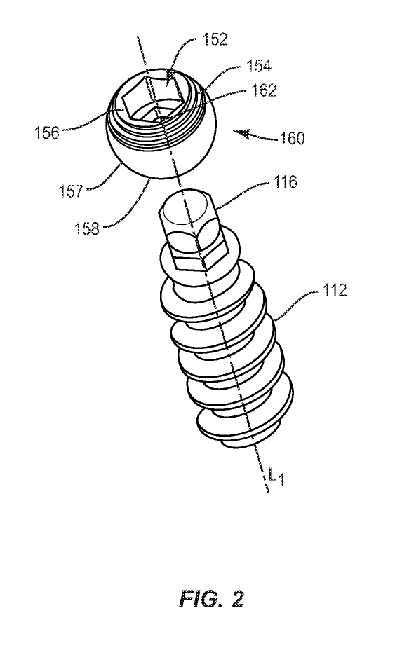

With further reference to FIGS. 2-4, in various embodiments, spherical connector 150 has a truncated spherical shape, is threaded with external threads 154 and is configured to fit into receptacle 134 at the distal end portion 128 of receiver 120. Spherical connector 150 is provided with an axial bore 152, an upper portion 156, a middle portion 157 and a lower portion 158. In some embodiments, from the upper portion 156 to the middle portion 157, and from the middle portion 157 to the lower portion 158, axial bore 152 is hexangularly shaped to fit onto the head 116 of the bone penetrating member 110. In various other embodiments, the axial bore can include many other shapes, for example, round, oval, rectangular, irregular, consistent, crescent, variable, uniform and non-uniform as required to fit over head 116 of bone penetrating member 110 in a lock and key fashion. In this manner, spherical connector 150 described in this disclosure can provide a universal attachment system to utilize a single bone screw component with multiple types of rod receivers, thereby significantly minimizing surgical inventory.

In some embodiments the middle portion 157 of spherical connector 150 includes notches 162 adapted to engage a corresponding engagement member such as inner lock ring 160, a ridge, internal thread or other feature of the spherical connector 150 to permit receiver 120 containing the spherical connector assembly to snap onto head 116 of bone penetrating member 110 when an axial force is applied to receiver 120 and locks onto bone penetrating member 110. In some embodiments, receiver 120 containing spherical connector 150 locks onto the head 116 of bone penetrating member 110 by means of a friction fitting. Once the spherical connector 150 is positioned into receptacle 134 of receiver 120, it is retained in place at distal end 128 by retaining member(s) 142.

Receiver 120 of spinal construct 100 can freely rotate about head 116 before a vertebral rod is secured within the spinal construct with a set screw and therefore head 116 is considered multi-axial. Once the spinal rod is captured within the slot or windows 138 of receiver 120 by the set screw, the vertebral rod is firmly pressed against interface connector 140 which, in turn, presses against spherical connector 150 which has firmly captured head 116 of bone penetrating member 110 within receiver 120 to prevent further movement of the receiver with respect to the shank 112.

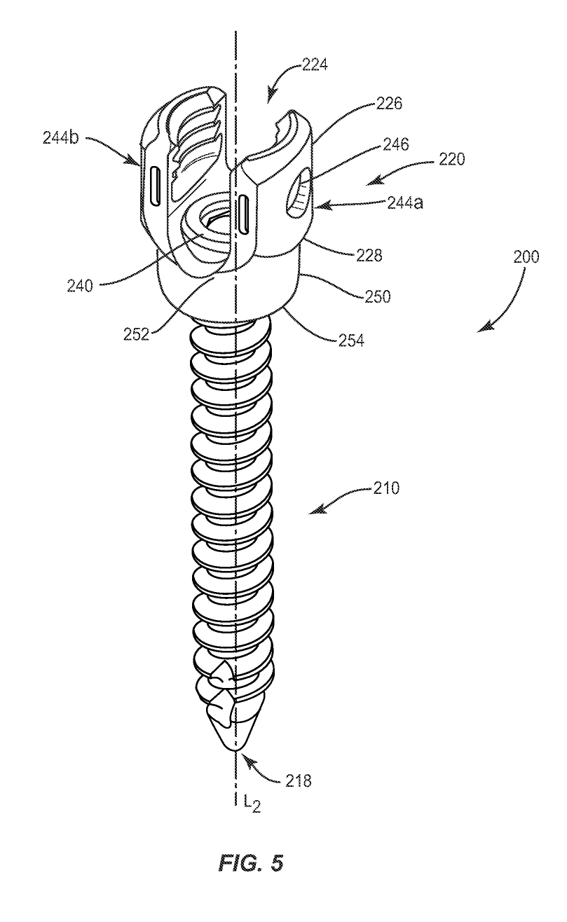

Spherical connector 150 (shown in FIG. 4) may be placed within many different kinds of receivers. With further reference to FIG. 5, in another embodiment, receiver 220 includes a body defining a longitudinal axis L₂, and having upright arms 244a and 244b, which are spaced apart so as to define an upper passage portion 224, to receive a vertebral rod in a lateral orientation. It is understood that the rod may have a number of desired lengths and diameters. In that regard, the width of the upper passage portion 224 in the current embodiment is substantially equal to the diameter of the rod member. In some embodiments, the width of the upper passage portion is slightly larger than the diameter of the rod, which allows easier insertion of the rod into the upper passage portion, allows for contouring of the vertebral rod, and also allows a variety of bone penetrating members of differing sizes to be used with receiver 220. Generally, the vertebral rod is positioned above the bottom portion of the upper passage portion 224 when in a locked position. However, in some embodiments the rod may be seated within the bottom portion of the upper passage portion 224 when in a locked position. Thus, the bottom portion of the upper passage portion 224 may be shaped or otherwise include features to ensure secure placement of the bone penetrating member, which penetrates bone using tip 218.

The upright arms 244a and 244b of the receiver 220 include a bore or hole 246, which extends through the upright arms. The holes 246 are substantially aligned with one another and are substantially perpendicular to the upper passage portion 224. In some instances, the hole 246 is utilized for grasping by a surgical tool to facilitate positioning of the rod into the spine of a patient. In an embodiment the upright arms 244a and 244b can have a tapered outer surface as they extend upwardly. This tapered outer surface reduces the bulk and size of the receiver 220 allowing for easier handling. In that regard, a surgical instrument may engage the holes 246 without substantially increasing the overall width needed to insert the spinal construct assembly.

Spinal construct 200 further includes a base 250, which is rotatably mounted to the bottom of receiver 220. Base 250 includes a generally cylindrical disk shaped body having an upper end 252 and a lower end 254. Base 250 includes a circumferential notch (not shown) adapted to engage a corresponding engagement member such as a ridge, snap ring, internal thread or other feature of the receiver 220 to permit relative respective rotation without separation of the base 250 and the receiver 220. Base 250 includes an inner wall surface (not shown) defining an interior cavity (not shown) in which the rounded head 216 of the bone penetrating member 210 is supported. The engagement between the proximal head 116 (of FIG. 3) and the interior cavity of the base is such as to permit rotation of the bone penetrating member 210 around axis L₂ and articulation of the bone penetrating member 210 around axis L₂.

Base 250 and, in some embodiments, also an area where upright arms 244a and 244b join the base are configured for receiving and retaining spherical connector 150 (shown in FIG. 4) therein. Spherical connector 150 (shown in FIG. 4) has the same configuration as described above in connection with FIGS. 2 to 4. In various embodiments an interface connector 240 is configured for receiving and retaining the upper portion of spherical connector 150 (shown in FIG. 4) therein. In various embodiments, interface connector 240 is shaped as an annular jacket in contact with and adjacent spherical connector 150 (shown in FIG. 4). In some embodiments it is contemplated that interface connector 240 minimizes potential friction between the vertebral rod and the spherical connector 150 (shown in FIG. 4). At its lower end, distal end portion 228 includes a retaining member(s) (not shown). In some embodiments retaining member(s) can be a washer or a ring.

In various embodiments, base 250 also includes an interior circumferential bearing surface circumferentially disposed about and configured to house spherical connector 150 (shown in FIG. 4). The bearing surface is connected to an interior wall surface (not shown) to define an interior cavity of the base in which spherical connector 150 (shown in FIG. 4) is disposed and adapted to receive the head 116 (shown in FIG. 3) of bone penetrating member 210.

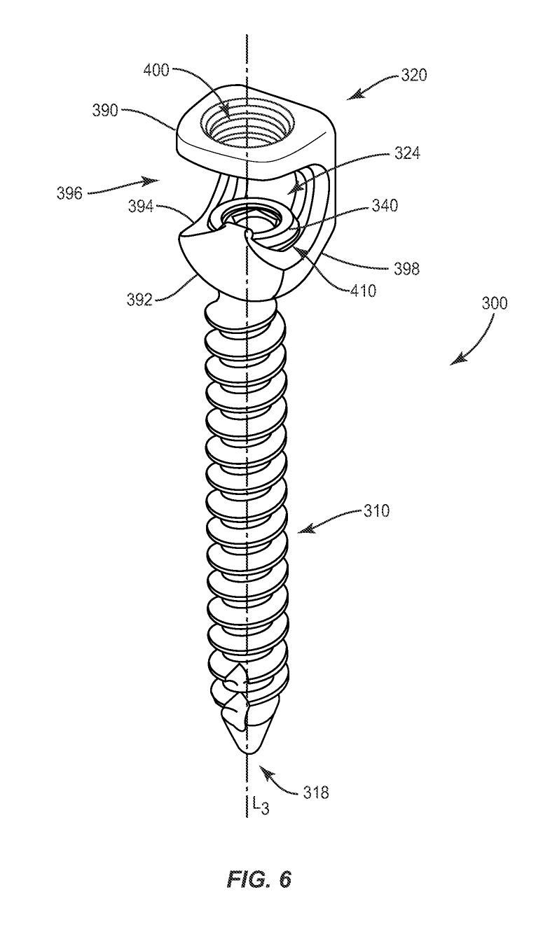

With further reference to FIG. 6, in yet another embodiment, spherical connector 150 (shown in FIG. 4) may be utilized in a spinal construct 300 having a C shaped receiver. Spinal construct 300, in the embodiment shown in FIGS. 6, includes an elongated bone penetrating member 310 extending from head (not shown) along a longitudinal axis L₃, to tip 318 of bone penetrating member 310, a receiver 320 containing an interface connector 340 in contact with and adjacent to a truncated spherical connector 150 (shown in FIG. 4) also contained in receiver 320.

Receiver 320 is substantially C-shaped, having an upper leg 390, a lower leg 392 including foot portion 394 extending from one end thereof, and an intermediate portion 396 joining upper and lower legs 390, 392 opposite of foot portion 394. Receiver 320 defines a mouth 398 between upper leg 390 and foot portion 394 that is opposite intermediate portion 396. Mouth 398 opens into upper passage portion 324 extending through receiver 320, with upper passage portion 324 extending in an orthogonal relationship to longitudinal axis L₃. Upper leg 390 has a threaded aperture 400 into which an engaging member, for example a set screw can be threadingly engaged.

In an embodiment, receiver 320 includes a cavity 410 in lower leg 392 and intermediate portion 396. Cavity 410 can be substantially cylindrical, extend from lower leg 392 along a distal portion of intermediate portion 396 and configured to receive truncated spherical connector 150 (shown in FIG. 4). Spherical connector 150 (shown in FIG. 4) is configured as shown and described in connection with FIGS. 2 to 4 and is in contact with and adjacent to interface connector 340. In this arrangement, an engaging member such as a set screw can be positioned to secure and direct a vertebral rod toward lower leg 392 and intermediate portion 396 and against interface connector 340 and spherical connector 150 (shown in FIG. 4).

In assembly, operation and use, the vertebral rod system including spinal construct 100, 200 or 300 is employed with a surgical procedure for treatment of a spinal disorder affecting a section of a spine of a patient, as discussed herein. The spinal construct 100, 200 or 300 may also be employed with other surgical procedures. It is contemplated that the vertebral rod system including spinal construct 100, 200 or 300 is attached to spinal vertebrae for fusion and/or dynamic stabilization applications of the affected section of the spine to facilitate healing and therapeutic treatment, while providing flexion, extension and/or torsion capabilities.

In use, to treat the affected section of the spine, a medical practitioner obtains access to a surgical site including spinal vertebrae in any appropriate manner, such as through incision and retraction of tissues. It is envisioned that the vertebral rod system including spinal construct 100, 200 or 300 may be used in any existing surgical method or technique including open surgery, mini-open surgery, minimally invasive surgery and percutaneous surgical implantation, whereby spinal vertebrae are accessed through a micro-incision, or sleeve that provides a protected passageway to the area. Once access to the surgical site is obtained, the particular surgical procedure is performed for treating the spinal disorder. The vertebral rod system including spinal construct 100, 200 or 300 is then employed to augment the surgical treatment. The vertebral rod system including spinal construct 100, 200 or 300 can be delivered or implanted as a pre-assembled device or can be assembled in situ. The vertebral rod system may be completely or partially revised, removed or replaced, for example, replacing rod and/or one or all of the components of spinal construct 100, 200 or 300.

Spinal construct 100, 200 or 300 may be employed as a bone screw, pedicle screw or multi-axial screw used in spinal surgery. It is contemplated that spinal construct 100, 200 or 300 may be coated with an osteoconductive material such as hydroxyapatite and/or osteoinductive agent such as a bone morphogenic protein for enhanced bony fixation. Spinal construct 100, 200 or 300 can be made of radiolucent materials such as polymers. Radiomarkers may be included for identification under x-ray, fluoroscopy, CT or other imaging techniques. Metallic or ceramic radiomarkers, such as tantalum beads, tantalum pins, titanium pins, titanium endcaps and platinum wires can be used, such as being disposed at the end portions of vertebral rod.

It is envisioned that the vertebral rod system described above including spinal construct 100, 200 or 300 may be employed with a vertebral rod having an arcuate configuration and an increased length providing the ability to extend over two or more intervertebral elements. It is contemplated that the configuration of the vertebral rod system may provide load sharing, dynamic and/or flexible stabilization over a plurality of intervertebral levels, including treated and untreated vertebral and intervertebral levels.

In one embodiment, the spinal construct includes an agent, which includes a bone growth promoting material, which may be disposed, packed or layered within, on or about the components and/or surfaces thereof. The bone growth promoting material, such as, for example, bone graft can be a particulate material, which may include an osteoconductive material such as hydroxyapatite and/or an osteoinductive agent such as a bone morphogenic protein (BMP) to enhance bony fixation of spinal construct 100, 200 or 300 with the adjacent vertebrae.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A spinal construct comprising:
a bone penetrating member having a head and a tip opposite the head,
a receiver extending along a longitudinal axis between a proximal end portion and a distal end portion of the receiver, the proximal end portion configured to receive an elongated member and the distal end portion comprising an opening for receiving
a spherical connector, the spherical connector configured to receive the head of the bone penetrating member.

2. A spinal construct of claim 1, wherein the spherical connector comprises a truncated spherical shape having an axial bore through an upper portion, a middle portion and a lower portion of the spherical connector, the upper portion further containing external ridges configured to fit into the distal end portion of the receiver.

3. A spinal construct of claim 2, wherein the upper portion and the lower portion of the spherical connector comprise a hexagon, a circle, an ellipse, a rectangle or a square shape.

4. A spinal construct of claim 2, wherein the spherical connector further comprises notches at the middle portion, the notches configured for receiving an inner locking ring.

5. A spinal construct of claim 2, wherein the receiver further comprises an interface connector and a retaining member, the interface connector for retaining the upper portion of the spherical connector, and the retaining member for retaining the lower portion of the spherical connector.

6. A spinal construct according to claim 1, wherein the receiver is configured to rotate freely about the head of the bone penetrating member prior to affixing the elongated member in the spinal construct.

7. A spinal construct according to claim 1, wherein the elongated member is a vertebral rod.

8. A spinal construct according to claim 1, further comprising a marker disposed on the spinal construct for indicating the position of the construct.

9. A spinal construct comprising:
a bone penetrating member having a head and a tip opposite the head,
a receiver extending along a longitudinal axis between a proximal end portion and a distal end portion of the receiver, and
a base having an upper end and a lower end, the base rotatably mounted to the distal end portion of the receiver, wherein the proximal end portion of the receiver is configured to receive an elongated member and (i) the base is configured to receive a spherical connector or (ii) the distal portion the receiver is configured to receive a spherical connector or (iii) the distal portion of the receiver and the base are configured to receive a spherical connector, the spherical connector adapted to receive the head of the bone penetrating member.

10. A spinal construct of claim 9, wherein the spherical connector comprises a truncated spherical shape having an axial bore through an upper portion, a middle portion and a lower portion of the spherical connector, the upper portion of the spherical connector further containing external ridges configured to fit into the base or the distal end portion of the receiver.

11. A spinal construct of claim 10, wherein the spherical connector further comprises notches at the middle portion, the notches configured for receiving an inner locking ring.

12. A spinal construct of claim 10, wherein the distal portion end of the receiver further comprises an interface connector and the base further comprises a retaining member, the interface connector for retaining the upper portion of the spherical connector, and the retaining member for retaining the lower portion of the spherical connector.

13. A spinal construct of claim 10, wherein the receiver is configured to rotate freely about the head of the bone penetrating member prior to affixing the elongated member in the spinal construct.

14. A spinal construct of claim 10, further comprising a marker disposed on the spinal construct for indicating the position of the construct.

15. A spinal construct comprising:
a bone penetrating member having a head and a tip opposite the head,
a receiver extending along a longitudinal axis between a proximal end portion and a distal end portion of the receiver, the receiver comprising:
an upper leg and a lower leg opposite the upper leg, the lower leg including a foot portion extending from one end thereof, the upper leg extending transversely to the longitudinal axis; an intermediate portion opposite the foot portion, the intermediate portion extending between the upper leg and the lower leg, the lower leg and the intermediate portion defining a cavity configured to receive a spherical connector, the spherical connector configured to receive the head of the bone penetrating member.

16. A spinal construct of claim 15, wherein the receiver further comprises an upper passage portion extending through the receiver substantially perpendicular to the longitudinal axis into which a spinal rod can be placed.

17. A spinal construct of claim 15, wherein the spherical connector comprises a truncated spherical shape having an axial bore through an upper portion, a middle portion and a lower portion of the spherical connector, the upper portion further containing external ridges configured to fit into the distal end portion of the receiver.

18. A spinal construct of claim 15, wherein the spherical connector further comprises notches at the middle portion, the notches configured for receiving an inner locking ring.

19. A spinal construct of claim 15, wherein the receiver further comprises an interface connector positioned in the cavity defined by the lower leg and the intermediate portion of the receiver and a retaining member, the interface connector for retaining the upper portion of the spherical connector, and the retaining member for retaining the lower portion of the spherical connector.

20. A spinal construct of claim 15, further comprising a marker disposed on the spinal construct for indicating the position of the construct.
